(19) United States
(12) Patent Application Publication
(10) Pub. No.: US 2021/0390595 A1
(43) Pub. Date: Dec. 16, 2021

(54) ELECTRONIC REPUTATION MANAGEMENT

(71) Applicant: PETER L. REX, Bellevue, WA (US)

(72) Inventor: PETER L. REX, Bellevue, WA (US)

(21) Appl. No.: 17/243,426

(22) Filed: Apr. 28, 2021

Related U.S. Application Data
(60) Provisional application No. 62/704,228, filed on Apr. 28, 2020.

(51) Int. Cl. G06Q 30/02

(52) U.S. Cl. CPC G06Q 30/0282; G06Q 30/0283

(57) ABSTRACT

At least one computer-readable medium on which are stored instructions that, when executed by one or more processing devices, enable the one or more processing devices to perform a method. The method includes the steps of receiving and storing in a database respective ratings of the performance of a service by a plurality of service providers, receiving from a first party a request for performance of the service; determining characteristics defining the request, based on the performance ratings and the characteristics defining the request, determining the optimal service provider of the plurality of service providers to fulfill the request, and assigning the optimal service provider to fulfill the request.

### PRIORITY CLAIM

This application claims priority from U.S. Provisional Application Ser. No. 62/704,228 filed Apr. 28, 2020, the entirety of which is hereby incorporated by reference as if fully set forth herein.

### BACKGROUND

Notably, previous attempts at a reputation management system in the field of contracting/property services and services/goods delivery have not been effective because they have taken, for example, a one size fits all work score, which rests on a false assumption that humans can be reduced in totality in their creative space of work to a single score.

### DRAWING FIGURES

FIG. 1 is a schematic view of an exemplary operating environment in which an embodiment of the invention can be implemented;

FIG. 2 is a functional block diagram of an exemplary operating environment in which an embodiment of the invention can be implemented;

FIG. 3 is a schematic illustration of the capture of professional service provider ratings according to an embodiment of the invention;

FIG. 4 is a schematic illustration of a selection engine according to an embodiment of the invention; and

FIG. 5 is a functional block diagram of an exemplary operating environment in which an embodiment of the invention can be implemented.

### DETAILED DESCRIPTION

This patent application is intended to describe one or more embodiments of the present invention. It is to be understood that the use of absolute terms, such as “must,” “will,” and the like, as well as specific quantities, is to be construed as being applicable to one or more of such embodiments, but not necessarily to all such embodiments. As such, embodiments of the invention may omit, or include a modification of, one or more features or functionalities described in the context of such absolute terms.

Embodiments of the invention may be described in the general context of computer-executable instructions, such as program modules, being executed by a processing device having specialized functionality and/or by computer-readable media on which such instructions or modules can be stored. Generally, program modules include routines, programs, objects, components, data structures, etc. that perform particular tasks or implement particular abstract data types. The invention may also be practiced in distributed computing environments where tasks arc performed by remote processing devices that arc linked through a communications network. In a distributed computing environment, program modules may be located in both local and remote computer storage media including memory storage devices.

According to one or more embodiments, the combination of software or computer-executable instructions with a computer-readable medium results in the creation of a machine or apparatus. Similarly, the execution of software or computer-executable instructions by a processing device results in the creation of a machine or apparatus, which may be distinguishable from the processing device, itself, according to an embodiment.

Correspondingly, it is to be understood that a computer-readable medium is transformed by storing software or computer-executable instructions thereon. Likewise, a processing device is transformed in the course of executing software or computer-executable instructions. Additionally, it is to be understood that a first set of data input to a processing device during, or otherwise in association with, the execution of software or computer-executable instructions by the processing device is transformed into a second set of data as a consequence of such execution. This second data set may subsequently be stored, displayed, or otherwise communicated. Such transformation, alluded to in each of the above examples, may be a consequence of, or otherwise involve, the physical alteration of portions of a computer-readable medium. Such transformation, alluded to in each of the above examples, may also be a consequence of, or otherwise involve, the physical alteration of, for example, the states of registers and/or counters associated with a processing device during execution of software or computer-executable instructions by the processing device.

As used herein, a process that is performed “automatically” may mean that the process is performed as a result of machine-executed instructions and does not, other than the establishment of user preferences, require manual effort.

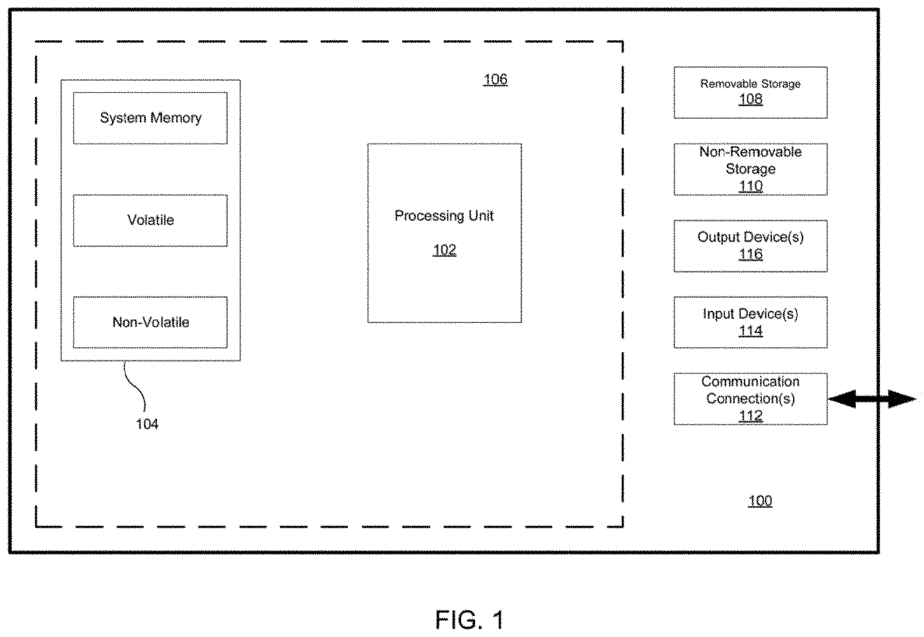

With reference to FIG. 1, an exemplary system for implementing an embodiment of the invention includes a computing device, such as computing device 100, which, in an embodiment, is or includes a smartphone. The computing device 100 typically includes at least one processing unit 102 and memory 104.

Depending on the exact configuration and type of computing device, memory 104 may be volatile (such as random-access memory (RAM)), nonvolatile (such as read-only memory (ROM), flash memory, etc.) or some combination of the two. This most basic configuration is illustrated in FIG. 1 by dashed line 106.

Additionally, the device 100 may have additional features, aspects, and functionality. For example, the device 100 may include additional storage (removable and/or non-removable) which may take the form of, but is not limited to, magnetic or optical disks or tapes. Such additional storage is illustrated in FIG. 1 by removable storage 108 and non-removable storage 110. Computer storage media includes volatile and nonvolatile, removable and non-removable media implemented in any method or technology for storage of information such as computer-readable instructions, data structures, program modules or other data. Memory 104, removable storage 108 and non-removable storage 110 are all examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by device 100. Any such computer storage media may be part of device 100.

The device 100 may also include a communications connection 112 that allows the device to communicate with other devices. The communications connection 112 is an example of communication media. Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, the communication media includes wired media such as a wired network or direct-wired connection, and wireless media such as acoustic, radio-frequency (RF), infrared, cellular and other wireless media. The term computer-readable media as used herein includes both storage media and communication media.

The device 100 may also have an input device 114 such as keyboard, mouse, pen, voice-input device, touch-input device, etc. Further, an output device 116 such as a display, speakers, printer, etc. may also be included. Additional input devices 114 and output devices 116 may be included depending on a desired functionality of the device 100.

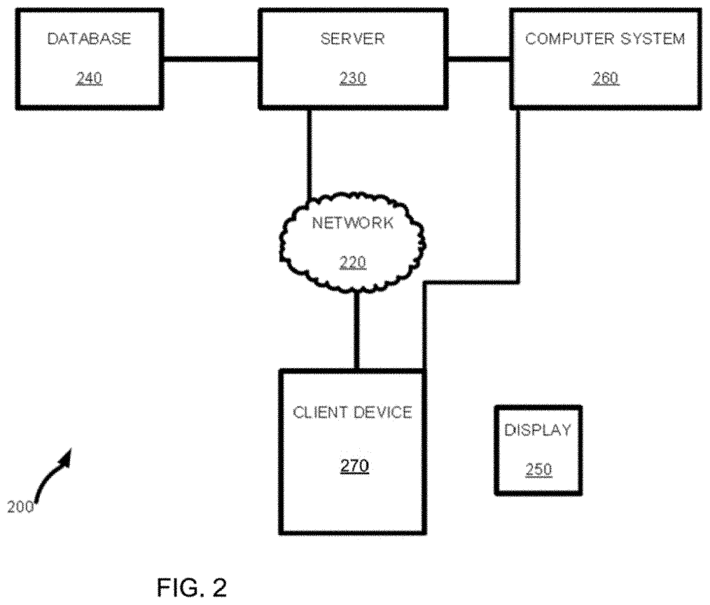

Referring now to FIG. 2, an embodiment of the present invention may take the form, and/or may be implemented using one or more elements, of an exemplary computer network system 200 that, in an embodiment, includes a server 230, database 240 and computer system 260. The system 200 may communicate with an electronic client device 270, such as a personal computer or workstation, tablet or smartphone, that is linked via a communication medium, such as a network 220 (e.g., the Internet), to one or more electronic devices or systems, such as server 230. The server 230 may further be coupled, or otherwise have access, to a database 240 and a computer system 260. Although the embodiment illustrated in FIG. 2 includes one server 230 coupled to one client device 270 via the network 220, it should be recognized that embodiments of the invention may be implemented using one or more such client devices coupled to one or more such servers.

The client device 270 and the server 230 may include all or fewer than all of the features associated with the device 100 illustrated in and discussed with reference to FIG. 1. The client device 270 includes or is otherwise coupled to a computer screen or display 250. The client device 270 may be used for various purposes such as network- and local-computing processes.

The client device 270 is linked via the network 220 to server 230 so that computer programs, such as, for example, a short message service (SMS) application, running on the client device 270 can cooperate in two-way communication with server 230. The server 230 may be coupled to database 240 to retrieve information therefrom and to store information thereto. Database 240 may have stored therein data (not shown) that can be used by the server 230 and/or client device 270 to enable performance of various aspects of embodiments of the invention. The data stored in database 240 may include, for example, information identifying customers, goods/services previously requested by such customers and providers of those goods/services. Additionally, the server 230 may be coupled to the computer system 260 in a manner allowing the server to delegate certain processing functions to the computer system. In an embodiment, most or all of the functionality described herein may be implemented in a desktop or smartphone application that may include one or more executable modules. In an embodiment, the client device 270 may bypass network 220 and communicate directly with computer system 260.

Previously, the reputation of service professionals, vendors, and contractors, particularly online, were one-dimensional. In actuality, true accurate and predictive reputation requires more details and attributes. An embodiment includes a process for rating the reputation of a vendor across many dimensions to include skills, job types, locations, property types, job cost profiles, job complexities, etc. to provide a robust scoring mechanism to enable accurate prediction of the quality of the work that the service professional will perform.

An embodiment provides a reputation management system in the field of contracting/property services and services/goods delivery more broadly. An embodiment of the invention provides quality control and satisfaction data provided by customers with regard to specific providers of goods or services (plus data received by the provider on the client/customer), in combination with existing vendor certification data—e.g., tradesman certifications—used to provide scoring on particular skills or capabilities pertaining to work. The data is used to route and match jobs or goods requested by a particular client (who also has workpoints) accurately to the right provider or professional.

Clients and professionals can also choose to showcase skill levels and competencies so as to boost their marketability, creating a unique personal brand for themselves in the new economy. For example, a painter of home interiors can choose to display the positive feedback data they receive for crown moulding and trim painting in order to attract more customers of crown-moulding- and trim-painting-specific projects to purchase their services. The painter could also use the same data to influence a tech-enabled general contractor to choose his services for projects fitting the same criteria.

One or more embodiments may employ the following technologies: multiple databases, mobile phones, algorithms, and machine learning.

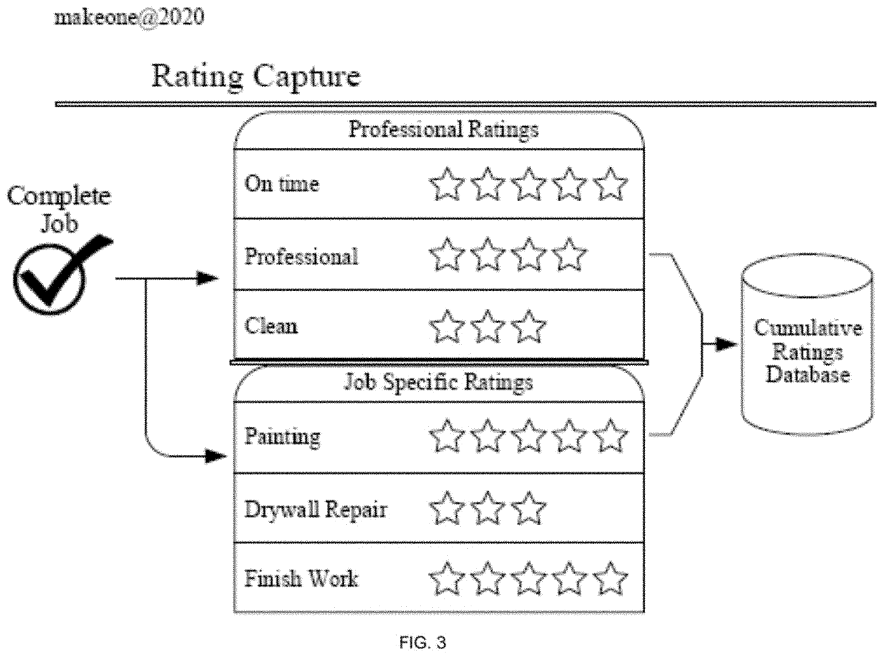

In a system and/or process according to an embodiment, which may employ a processing device and a memory, and referring to FIG. 3, a service or job is completed by a service professional. The customer and/or internal team members rate the professional's general attributes, such as the professional's punctuality, professionalism and cleanliness, and job/skill-specific attributes (some attributes are driven by job types). Ratings are stored and combined with historical ratings in a cumulative ratings database, such as database 240, to improve overall accuracy of the pro's various ratings.

In a system according to an embodiment, a service/job request is received by the system. The system collects attributes on the request (e.g., job type, location, property class, order history, pro availability, etc.). The system references the cumulative historical ratings database combined with the request attributes to identify the best professionals for the requested service. The system determines the punctuality of the pro using the GPS functionality of the pro's phone. For example, the system keeps track of the location of the pro at specific important times to assess or “grade” the extent to which the pro arrives at the service site on time according to the scheduled start time of the service.

The system captures the efficiency of the pro from start to finish of the service on-site using GPS location, which indicates how quick or slow the pro is in different jobs and or circumstances. For example, with the combination of location tracking and time, the system can track the times at which the pro is present at the service site, as well as their location(s) once they leave the site (e.g., to determine if they're purchasing materials need to perform the service). If it's a multi-day job, the system can record the comings and goings of the pro as well as the total time spent performing the service. All of this data can be analyzed to grade the quality of the pro on a number of dimensions (e.g., on time, professional, fast, etc.). Professionals can be manually selected from a suggested list or automatically assigned.

An embodiment collects job and skill ratings with every completed job as a standard part of completion approval workflows. This enables creation of a large database of performance ratings for professionals that use an embodiment.

With mobile, voice, and web interfaces, an embodiment can collect ratings from customers, vendors and partners and combine them in novel ways to create a rich view of a professional's work quality history. When the pro completes the job, the pro can enter into the system completion information, including self-rating of the job via text, web or voice. If the work is to be completed by multiple pros, each such pro may be incentivized to evaluate and provide feedback rating the work done by the other pros on the job. Additionally, pros who do subsequent work at the same service site also may be incentivized to evaluate and provide feedback rating the work done by their predecessor pros on the job. The customer may also be asked to review the work and provide feedback.

In order for ratings of pros to be valuable, the ratings must take into account the countless circumstances of the pros' work histories. These circumstances provide rich context and information about the quality of the pro, and also acknowledges that some pros can perform better or worse in different circumstances.

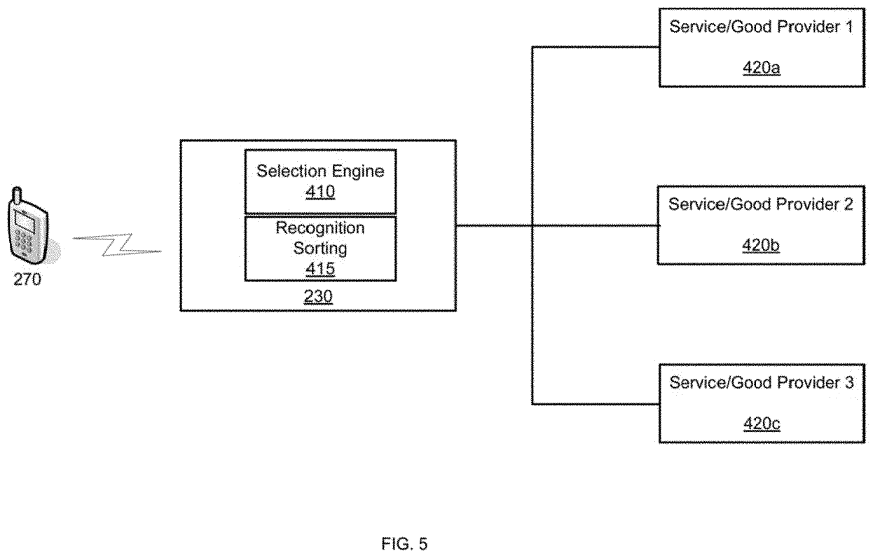

Referring to FIG. 5, and in an embodiment of the invention, a customer initiates an order for a good or service by sending an email, a voice call or an SMS message including text and/or a voice recording from a client device 270 to server 230 using a designated SMS number associated with server 230. In this embodiment, a recognition sorting application 415 executed by the server 230 monitors each email, call or SMS message incoming from client devices 270 and, based on one or more characteristics associated with the call and/or message, identifies the type of good or service requested by the customer. The one or more characteristics may include one or more keywords, textual or part of a voice recording, in the email, text message or voice call that indicate the type of good/service requested and that arc recognized by artificial intelligence associated with the recognition sorting application 415. Once the requested good/service has been identified by the recognition sorting application 415, and as explained in greater detail below, a selection engine 410 executed by the server 230 identifies an optimal service provider 420 from among a plurality of service providers by which the order should be fulfilled.

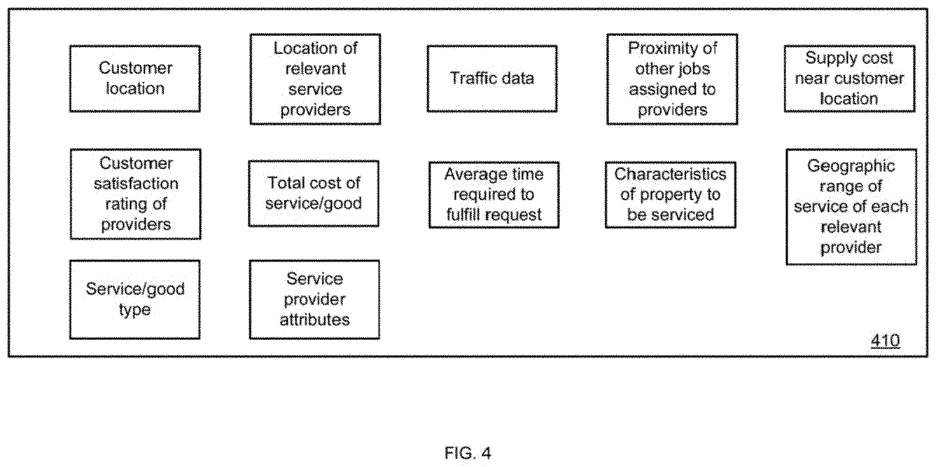

As best illustrated in FIG. 4, the selection engine 410 may determine a list of suitable service providers 420 ranked by scores assigned to each candidate service provider based on one or more combinations of the following non-exhaustive list of factors derived from data sets that may be stored in, for example, database 240:

Service/good type;

Location(s) of service(s) to be performed and/or customer, which may be determined using GPS data received from the customer's client device 270;

Location(s) of qualified professional(s);

Geographic location of areas the professionals arc willing to service;

Location(s) and/or cost of materials and tools required to fulfill customer request;

Estimated drive times required for professional to fulfill service;

Factors affecting transport times (e.g., weather forecast, projected traffic patterns, etc.);

Service attributes (e.g., after-hours work, dollar amount of work, work duration, etc.);

Professional attributes (e.g., reputation, skill ratings, professional ratings, etc.);

Attributes of property/location to be serviced (e.g., parking onsite, office check-in, after hours work approved, etc.).

The server 230 through wired or wireless means may then offer the requested service transaction to each of the listed service providers in descending order of rank until the highest-ranked provider accepts the transaction.

The routing and sorting functions may be performed on respective different servers or on the same server such as server 230. In an embodiment, the routing and sorting functions are cloud-based services that also integrate with the service request and fulfillment system.

For example, John the Pro is a general handyman who has performed hundreds of jobs in the Austin metro area over the past three years. He has done many types of jobs including roof and gutter repairs, fence repairs, drywall fixes, brick work, patio and deck repairs. Data tied to these jobs include information on whether the job was an emergency or normally scheduled work. Data tied to these jobs also include information on where the jobs were located, the type of property (single family, condos/apartment). Data tied to location includes additional information about the weather at the time of the work.

With all of this information about John the Pro, the system can construct deeper “dimensions of quality” about John's work for the purposes of routing work to him that better matches his strengths, such as:

John is very punctual for emergency work.

John tends to be late to jobs that are in North Austin.

John is on time when the weather is sunny and warm.

John gets work done faster than average when the jobs are outdoors vs. indoors.

John's work on multi-family properties gets rated higher than on single family homes.

John's drywall work quality is very high, but his roof and gutter repair work is below average.

For job types that arc more complex, John takes much longer than average to complete the job.

These deep and valuable scores can be used to provide a selection of the best professionals for job routing (automated and/or human powered).

While the preferred embodiment of the invention has been illustrated and described, as noted above, many changes can be made without departing from the spirit and scope of the invention. Accordingly, the scope of the invention is not limited by the disclosure of the preferred embodiment. Instead, the invention should be determined entirely by reference to the claims that follow.

What is claimed is:

1. At least one computer-readable medium on which are stored instructions that, when executed by one or more processing devices, enable the one or more processing devices to perform a method, the method comprising the steps of:
receiving and storing in a database respective ratings of the performance of a service by a plurality of service providers;
receiving from a first party a request for performance of the service;
determining characteristics defining the request;
based on the performance ratings and the characteristics defining the request, determining the optimal service provider of the plurality of service providers to fulfill the request; and
assigning the optimal service provider to fulfill the request.

2. The medium of claim 1, wherein the characteristics comprise one or more textual keywords.

3. The medium of claim 1, wherein the characteristics comprise one or more spoken keywords.

4. The medium of claim 1, wherein the performance ratings are based on a geographic location of the optimal service provider.

5. The medium of claim 1, wherein the performance ratings are based on a geographic location of an area in which the optimal service provider is willing to fulfill the request.

6. The medium of claim 1, wherein the performance ratings are based on one of a location and cost of supplies required to fulfill the request.

7. The medium of claim 1, wherein the performance ratings are based on an estimated drive time required for the optimal service provider to fulfill the request.

8. The medium of claim 1, wherein the performance ratings arc based on projected traffic patterns proximal to a location of the optimal service provider.

9. The medium of claim 1, wherein the performance ratings arc based on a customer rating of the optimal service provider.
